(19) United States
(12) Patent Application Publication
(10) Pub. No.: US 2021/0274855 A1
(43) Pub. Date: Sep. 9, 2021

(54) INSULIN PUMP COMPATIBLE UNDERGARMENTS

(71) Applicant: Lauren Anne Hermsen, Chicago, IL (US)

(72) Inventor: Lauren Anne Hermsen, Chicago, IL (US)

(21) Appl. No.: 17/249,628

(22) Filed: Mar. 8, 2021

Related U.S. Application Data
(60) Provisional application No. 62/986,873, filed on Mar. 9, 2020.

(51) Int. Cl. A41B 9/00; A61M 5/142; A41C 3/00

(52) U.S. Cl. CPC A41B 9/001; A41B 2400/38; A41C 3/0035; A61M 5/14248

(57) ABSTRACT

An insulin pump compatible undergarment has a pocket to contain an insulin pump and an access port to accommodate medical tubing. The pocket has an opening overlapped with a pocket edge. A method of wearing an insulin pump includes donning the undergarment; inserting an insulin pump into the overlapped pocket; feeding medical tubing through the access port; coupling the medical tubing with the insulin pump; and coupling the medical tubing with a cannula inserted at an infusion site.

### CROSS-REFERENCE TO RELATED APPLICATION

This application claims the benefit of priority of U.S. provisional application No. 62/986,873, filed Mar. 9, 2020, the contents of which are herein incorporated by reference.

### BACKGROUND OF THE INVENTION

The present invention relates to undergarments with storage and accessibility for medical devices and, more particularly, to undergarments with insulin pump accessibility.

Many diabetics, including Type 1 diabetics, wear an insulin pump to help keep their blood glucose level balanced. The pump must be accessible throughout the day. Existing garments often leave the diabetic struggling to figure out where to put the insulin pump that will keep it safe and out of the way, while also keeping it easily accessible at the same time. While a compartment may be available in some garments, the garment compartment is not positioned to allow easy access to the pump. Moreover, the position of the garment compartment results in bending or kinking of the tubing which disrupts the flow of insulin and may cause serious harm.

As can be seen, there is a need for undergarments with compartments that will keep an insulin pump safe and accessible without bending the tubing.

### SUMMARY OF THE INVENTION

The present invention provides undergarments comprising strategically placed pockets with access ports so that insulin pump tubing may exit the pocket without bending or kinking (i.e., causing disruption to insulin flow). The pockets are placed to optimize access to where potential pump sites might be, considering length of tubing and accessibility. The inventive undergarments provide easy access to the pump while also keeping it in a location that is protected and out of the way.

The pockets may also be used to hold credit cards, IDs, keys, and the like.

In one aspect of the present invention, an insulin pump compatible undergarment is provided, comprising an undergarment having a pocket and at least one access port; wherein the pocket has an opening overlapped with a pocket edge; wherein the pocket is operative to contain an insulin pump; and wherein the at least one access port is operative to accommodate medical tubing.

In another aspect of the present invention, a method of wearing an insulin pump is provided, comprising: donning an undergarment having an overlapped pocket with at least one access port at a corner thereof; inserting an insulin pump into the overlapped pocket; feeding a medical tubing through the at least one access port; coupling the medical tubing with the insulin pump; and coupling the medical tubing with a cannula inserted at an infusion site.

These and other features, aspects and advantages of the present invention will become better understood with reference to the following drawings, description, and claims.

### BRIEF DESCRIPTION OF THE DRAWINGS

FIG. 1 is a perspective view of an undergarment according to an embodiment of the present invention, shown in use;

FIG. 2 is a detail view thereof, taken from line 2-2 of FIG. 1, showing the front of the pocket broken away;

FIG. 3 is a sectional view thereof, taken along line 3-3 of FIG. 2;

FIG. 4 is a sectional view thereof, taken along line 4-4 of FIG. 2;

FIG. 5 is a perspective view of an undergarment according to another embodiment of the present invention, showing the pocket in use; and

FIG. 6 is a perspective view of an undergarment according to another embodiment of the present invention, showing the pocket in use.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is an undergarment with an overlapping pocket operative to contain an insulin pump. Each pocket is provided with at least one access port or hole at a corner of the pocket to accommodate medical tubing for the insulin pump. The pockets are placed on each undergarment at a predetermined position selected to prevent bending or kinking of the tubing. The pocket placement is not particularly limited.

As used herein, the terms “overlapped pocket”, “overlapping pocket”, and “pocket with an overlapping edge” are used interchangeably to describe a pocket having an opening over which a portion of the fabric that forms the pocket, also referred to as a “pocket edge”, folds.

The type of inventive undergarment is not particularly limited, provided that the undergarment fits closely against the user's skin to minimize movement of an insulin pump and provided that the garment has sufficient material to accommodate a pocket having a size comparable to the pump. The undergarment may be, for example, shapewear and/or a brassiere such as a long line bralette or sports bra. As used herein, the term “shapewear” is not limited to a garment designed to make the user appear slimmer. Rather, “shapewear” refers to any garment worn on the hips and thighs that fits closely against the user's skin.

The inventive undergarments and the pockets or compartments thereof may be manufactured from any suitable materials. Polyester and spandex as well as nylon and spandex are common blends for undergarments, providing comfort and support.

To prevent shifting and rolling and to keep the insulin pump in place, edges of the undergarments and interior surfaces of the compartments thereon may be lined with a securing material that provides traction against the user's skin, such as premium gripper elastic or an adhesive. For example, gripper elastic may be present on an interior surface of the undergarment co-located with the pocket.

The pocket on bras may be placed, for example, centered under the breasts.

The pockets on shapewear may be provided, for example, on both inner thighs so that either pocket may be used, depending upon which is closer to the pump site on that day. The shapewear may be made with overlapping edge pockets which may be lined with a securing material.

A long line bralette with a wide band around the torso, for example, about 4-inches in width, may have a pocket placed within the wide band (i.e., centered under the breasts). The pocket may be constructed with an overlapping edge as well as a securing material to help keep the pump secured. Pockets may also have ports cut in the corners so that the tubing can exit without being bent or kinked.

A method of using the inventive undergarment is as follows. The user may simply put on (don) the bralette or shapewear as the user normally would wear currently available undergarments and secure the pump in a pocket either on the bra or on the shapewear selected by the user. Once the pump is inserted into the pocket, the user may feed the tubing through a port in the pocket and reattach the tubing to a cannula at the user's infusion site so that the tubing is coupled to both the pump and the cannula. Alternatively, the tubing may be fed through the port and attached to the pump before the pump is inserted into the pocket. The inventive undergarment eliminates the need to search for a place to hook the pump.

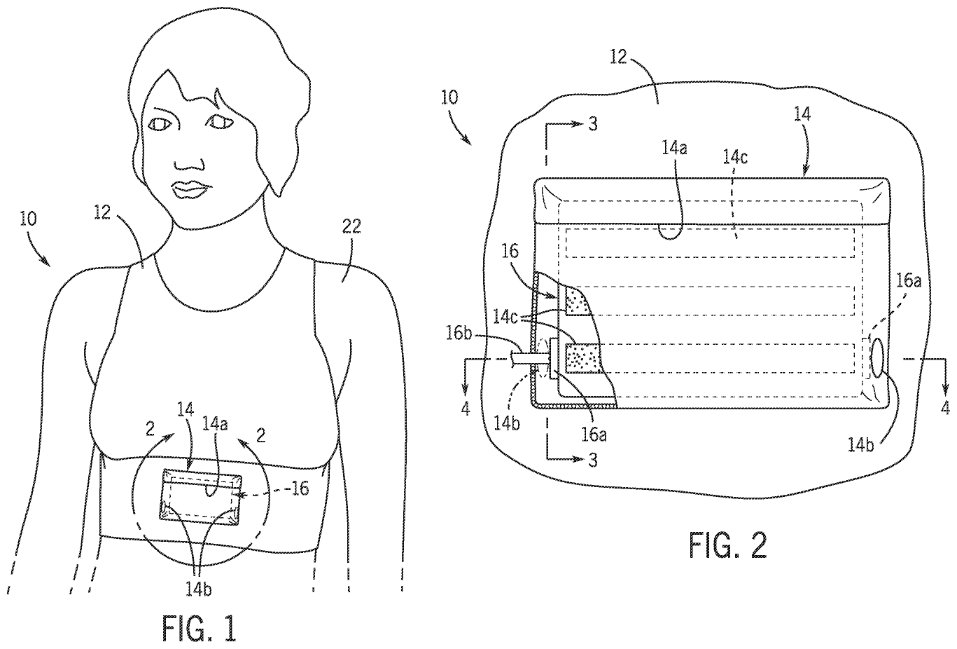

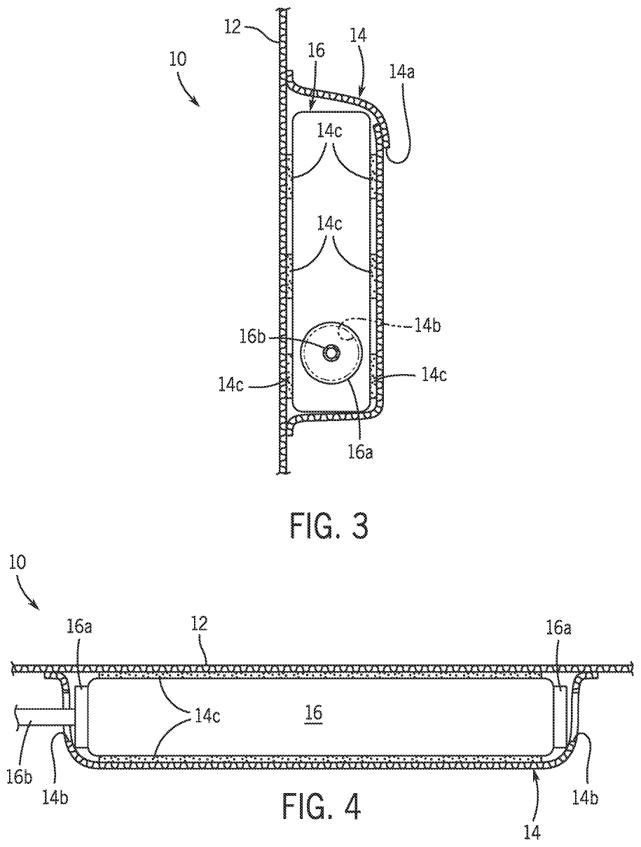

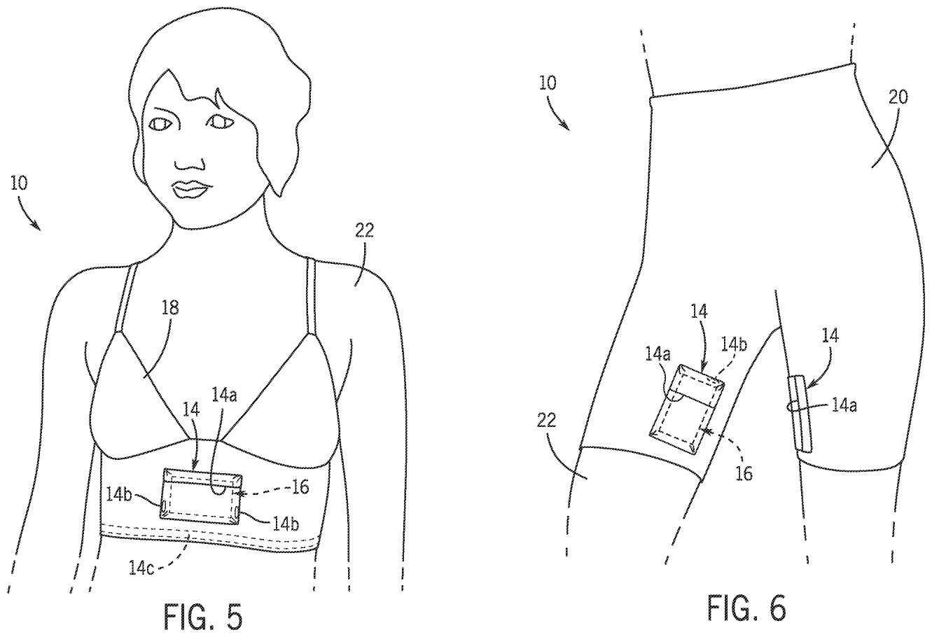

Referring to FIGS. 1 through 6, FIG. 1 shows an insulin pump compatible undergarment 10 according to an embodiment of the present invention comprising a sports bra 12 with a pocket 14. As shown in FIG. 2, the pocket 14 may have an overlapping edge 14a and securing strips 14c to ensure secure placement of an insulin pump 16. A tube 16b attached to an insulin pump port 16a on the pump 16 may be fed through an access port 14b on the pocket 14. The invention is not limited to a sports bra and may include any type of undergarment, such as a bralette 18 and/or shapewear 20. The pocket 14 may be positioned anywhere on the undergarment 10 that provides easy access to the user's 22 infusion site without bending or kinking the tubing. In the embodiments of FIGS. 1 and 5, the pocket 14 is shown centered beneath the cups. The shapewear 20 of FIG. 6 is shown with a pocket 14 on each inner thigh.

It should be understood, of course, that the foregoing relates to exemplary embodiments of the invention and that modifications may be made without departing from the spirit and scope of the invention as set forth in the following claims.

What is claimed is:

1. An insulin pump compatible undergarment, comprising an undergarment having a pocket and at least one access port; wherein the pocket has an opening overlapped with a pocket edge; wherein the pocket is operative to contain an insulin pump; and wherein the at least one access port is operative to accommodate medical tubing.

2. The insulin pump compatible undergarment of claim 1, wherein the undergarment is shapewear with the pocket on an inner thigh.

3. The insulin pump compatible undergarment of claim 1, wherein the undergarment is a brassiere with cups and the pocket is centered below the cups.

4. The insulin pump compatible undergarment of claim 1, further comprising a securing material on at least one edge of the undergarment and on an interior surface of the undergarment co-located with the pocket.

5. The insulin pump compatible undergarment of claim 1, wherein an interior surface of the pocket has securing material.

6. The insulin pump compatible undergarment of claim 1, wherein the at least one access port is provided at a corner of the pocket.

7. The insulin pump compatible undergarment of claim 1, further comprising a securing material on at least one edge of the undergarment, on an interior surface of the undergarment co-located with the pocket, and on an interior surface of the pocket, wherein the at least one access port is provided at a corner of the pocket.

8. A method of wearing an insulin pump, comprising:
a) donning an undergarment having an overlapped pocket with at least one access port at a corner thereof;
b) inserting an insulin pump into the overlapped pocket;
c) feeding a medical tubing through the at least one access port;
d) coupling the medical tubing with the insulin pump; and
e) coupling the medical tubing with a cannula inserted at an infusion site.

9. The method of claim 8, wherein the medical tubing is fed through the at least one access port and coupled to the insulin pump before the insulin pump is inserted into the overlapped pocket.

10. The method of claim 8, wherein the medical tubing is coupled to the insulin pump before feeding the medical tubing through the at least one access port.
